(19) United States
(12) Patent Application Publication
(10) Pub. No.: US 2026/0257056 A1
(43) Pub. Date: Sep. 3, 2026

(54) MODULAR ASSEMBLY OF IMPLANTABLE ELECTROMOTIVE MICROMECHANICAL BLOOD PUMP

(71) Applicant: Procyrion, Inc., Houston, TX (US)

(72) Inventors: Av Edidin, Houston, TX (US); Ronald G. Earles, Houston, TX (US); Omar M. Benavides, Houston, TX (US); Christopher A. Durst, Houston, TX (US)

(21) Appl. No.: 19/383,518

(22) Filed: Nov. 7, 2025

Related U.S. Application Data
(60) Provisional application No. 63/718,540, filed on Nov. 8, 2024.

(51) Int. Cl. A61M 60/88; A61M 60/139; A61M 60/216; A61M 60/419

(52) U.S. Cl. CPC A61M 60/88; A61M 60/139; A61M 60/216; A61M 60/419; A61M 2207/00

(57) ABSTRACT

A method for assembling a blood pump is provided. In the method, a first plurality of contacts of lead contact housing of a lead assembly is coupled with a second plurality of disposed on a first end of a motor housing of a drive unit. A free end of the lead contact housing is positioned adjacent to a surface of a motor housing. The lead contact housing is circumferentially joined to the motor housing, e.g., at the junction of the free end of the lead contact housing and the surface of the motor housing, while maintaining a position and/or orientation of the lead contact housing relative to the motor housing.

### INCORPORATION BY REFERENCE TO ANY PRIORITY APPLICATIONS

This application claims priority to U.S. Patent Application No. 63/718,540, filed on Nov. 8, 2024, the entire contents of which are incorporated by reference herein in their entirety and for all purposes. Any and all applications for which a foreign or domestic priority claim is identified in the Application Data Sheet as filed with the present application are hereby incorporated by reference under 37 CFR 1.57.

### BACKGROUND

### Field

The field relates to assembly methods for an implantable electromotive micromechanical blood pump.

### Description of the Related Art

In the field of cardiac assist devices and mechanical circulatory support, blood pumps are used to support the heart in circulating blood through the body. Some of these blood pumps are intravascular blood pumps and are designed or adapted for use within blood vessels.

### SUMMARY OF THE INVENTION

In one embodiment, a method for assembling a medical device is disclosed. The method can comprise providing a power delivery module configured to delivery electrical power during operation of the medical device; providing a drive module configured to convert the electrical power from the power delivery module to motion of a drive device during operation of the medical device; providing a hydraulic module comprising a treatment device, the motion of the drive device imparting motion to the treatment device during operation of the medical device; and connecting the drive module to the power delivery module and connecting the hydraulic module to the drive module.

In some embodiments, the treatment device comprises a pump. For example, the motion can comprise rotational motion. In other embodiments, the motion can comprise translational motion. For example, at least one of the drive device and the treatment device can comprise a piston. In some embodiments, the drive module is connected (e.g., welded) to the power deliver module before connecting the hydraulic module to the drive module (e.g., by welding). In some aspects, connecting the drive module to the power delivery module is performed after separately assembling the drive module and the power delivery module and wherein connecting the hydraulic module to the drive module is performed after separately assembling the drive module and the hydraulic module.

In another aspect, a method for assembling a medical device (e.g., a pump with impeller) is disclosed. For example, a method for assembling a blood pump is provided. A power delivery module is provided that comprises an elongate lead body. The elongate lead body has conductors configured to convey power therethrough from a first end configured to be electrically connected to a controller to a second end. The second end has a lead contact housing supporting a first plurality of contacts. The first plurality of contacts is coupled with a second plurality of contacts disposed on a first end portion of a motor housing of a drive module. A free end of the lead contact housing is positioned adjacent to a first surface of the motor housing. A free end of a shroud of a hydraulic module is positioned adjacent to a second surface of the motor housing. The lead contact housing is circumferentially joined to the motor housing while maintaining a position and/or orientation of the lead contact housing relative to the motor housing. The free end of the shroud is circumferentially joined to the motor housing while maintaining a position and/or orientation of the shroud relative to the motor housing.

In some embodiments, the first plurality of contacts comprises pins and the second plurality of contacts comprises sockets and wherein coupling the first plurality of contacts with the second plurality of contacts comprises inserting the pins of the power delivery module into the sockets of the drive module. In some aspects, upon positioning the free end of the lead contact housing against the first surface of the motor housing, the pins of the power module are in electrical contact with the sockets of the drive module. In some aspects, maintaining the position and/or orientation of the lead contact housing relative to the motor housing comprises aligning a longitudinal axis of the lead contact housing with a longitudinal axis of the motor housing. The first plurality of contacts comprises sockets and the second plurality of contacts can comprises pin and wherein coupling the first plurality of contacts with the second plurality of contacts can comprise advancing the sockets of the power delivery module over the pins of the drive module. In some embodiments, circumferentially joining the lead contact housing to the motor housing comprises providing relative motion between a welder and the lead contact housing and the motor housing while the position and/or orientation of the lead contact housing and the motor housing are maintained. In some embodiments, circumferentially joining the lead contact housing to the motor housing comprises providing relative rotational motion between a welder and the lead contact housing and the motor housing while the position and/or orientation of the lead contact housing and the motor housing are maintained. In some embodiments, circumferentially joining the motor housing to the shroud comprises providing relative rotational motion between a welder and the shroud and the motor housing while the position and/or orientation of the shroud and the motor housing are maintained. In some aspects, a hermetic seal is provided for the drive module by circumferentially joining the motor housing to the lead contact housing. In some aspects, an ionic seal is provided for the lead contact housing by circumferentially joining the motor housing to the lead contact housing. In some embodiments, the method provides a blood pump assembly having a width suitable for placement in an aorta of a patient. For example, the elongate motor housing and the lead contact housing can have a diameter configured to permit substantial blood flow between an outer surfaces thereof and an aorta when deployed. As an example, the elongate motor housing can have a diameter less than 2 cm. In some embodiments, the hydraulic module comprises an impeller magnetically rotatable by operation of a motor disposed in the drive module. In some embodiments, positioning the impeller of the hydraulic module within the shroud and wherein positioning the free end of the shroud of the hydraulic module adjacent to the second surfaced of the motor housing causes a magnet in the impeller to be magnetically coupled with a magnet in the drive module. In some embodiments, a cover of the motor housing of the drive module comprises a fixed projection, the impeller being disposed over the fixed projection when positioning the free end of the shroud of the hydraulic module adjacent to the second surfaced of the motor housing. In some embodiments, the cover comprises a continuous surface without any openings for an output shaft of the motor. In some embodiments, an output shaft of the motor is coupled with a magnet at a free end thereof, the magnet the free end being separated from an inside surface of a cover of the drive module. In some embodiments, a distal radial bearing is supported in the hydraulic module and further comprising positioning the impeller in the shroud and sliding a distal portion of a shaft of the impeller into the distal radial bearing prior to positioning the free end of the shroud of the hydraulic module adjacent to the second surface of the motor housing. In some embodiments, the hydraulic module comprises a strut module, the method further comprising joining the strut module to the shroud by forming at least one weld between a distal portion of the shroud and a sleeve coupled with the strut module. In some embodiments, the method includes engaging a radial projection of the sleeve with a recess formed in a distal surface a proximal ring of the strut module to couple sleeve with the strut module. In some embodiments, the proximal ring comprises a continuous unperforated surface extending entirely around the proximal ring, the sleeve being disposed within the inner surface of the proximal ring when coupled to the strut module and extending to a position within the distal portion of the shroud. In some embodiments, the method comprises positioning a printed circuit board in a passage of the motor housing, electrically connecting the second plurality of contacts with the printed circuit board, and fixing the position of the second contacts adjacent to the first surface of the motor housing. In some embodiments, the second plurality of contacts comprises press-fit receptacles configured to be deflected by engagement thereof with the first plurality of contacts.

In another aspect, a method for assembling a medical device (such as a blood pump) is provided. In the method, a first plurality of contacts of lead contact housing of a lead assembly is coupled with a second plurality of disposed on a first end of a motor housing of a drive unit. A free end of the lead contact housing is positioned adjacent to a first surface of a motor housing. The lead contact housing is circumferentially joined to the motor housing while maintaining a position and/or orientation of the lead contact housing relative to the motor housing.

In some embodiments, circumferentially joining comprises forming a laser weld about the periphery of the motor housing and the lead contact housing. In some embodiments, a hermetic seal is provided for the motor housing by circumferentially joining the motor housing to the lead contact housing. In some embodiments, an ionic seal is provided for the lead contact housing by circumferentially joining the motor housing to the lead contact housing. In some embodiments, the method includes: positioning a free end of a shroud disposed around an impeller adjacent to a second surface of the motor housing; and joining the free end of the shroud to the motor housing while maintaining a position and/or orientation of the shroud relative to the motor housing. In some embodiments, joining comprises circumferential joining. In some embodiments, circumferentially joining comprises forming a laser weld about the periphery of the motor housing and the shroud.

The foregoing methods can further include coupling a support or localization structure for a pump to the motor housing. The localization structure can be one that limits or prevents translation, limit or prevent rotation, aids in maintaining the position of some part of the pump relative to some anatomical structure, or any combination of these is needed. The localization structure may be designed for acute, semi-acute, semi-chronic, or chronic use.

Blood is a harsh environment for devices and any thrombus, foreign material, or pathogen in a blood vessel could have dire consequences. Novel localization structures that are biocompatible, non-thrombogenic, and non-hemolytic for well in excess of the expected duration of use are needed. Additionally, the function and removal of novel means of localization should preferentially be consistent with any endothelialization that may occur during the expected duration of use. Methods of securing localization structures facilitate operation and removal.

Novel localization structures for intravascular devices preferably provide biocompatibility of materials and surfaces, design for hemodynamic compatibility (reduced or minimal flow mediated thrombogenicity and hemolysis and disruption to natural flow), reduced or minimal trauma to the inside of the vessel or other anatomical structures, sufficient localization and freedom of motion, and removability when the therapy that the localized device provides is complete. Removability is facilitated by securing a strut module to the motor housing using a single weld step as discussed herein. Such a step can provide good mechanical integrity under loading applied for removal.

Localization and positioning systems and methods for medical devices, such as intravascular blood pumps or other intravascular devices are disclosed herein. The various embodiments comprise one or more of the following elements: struts extending from the device to be localized, said struts providing constant or intermittent contact with the vessel wall; a tether (e.g., a power lead) to limit translation and aid in positioning; and propulsion to maintain localization. Such struts can be coupled with a motor housing by mechanically capturing a connecting structure of a strut module including the struts and thereafter using a single welding step to circumferentially couple the connecting structure directly or indirectly the struts to the motor housing.

In some embodiments, the localization and positioning system may be part of or include a support structure that comprises struts that are projections that extend distally and radially outward from the device to contact the blood vessel walls or other anatomical features. Various illustrated embodiments show the struts extending distal the pump housing and impeller. However it should be appreciated that, alternatively, any of the struts may instead extend proximal the pump head (e.g., proximal the motor housing). In such embodiments, one or more of the struts can extend proximally from the drive unit or shroud. In yet other embodiments, one or more, e.g., a first plurality of struts can extend distal the pump housing and impeller, and one or more, e.g., a second plurality of struts can extend proximal the pump head (e.g., proximal the motor housing). The struts may be shaped, formed, and processed so that for a given outward radial force in the expanded configuration, the radial force in the collapsed configuration and/or the force to move from the expanded to collapsed configuration is reduced (e.g., minimized). The process of securing the struts to the motor housing can including coupling the struts with a structure more suited for welding to the motor housing.

Struts may consist of or otherwise be formed from a biocompatible metal, shape memory alloy, or alloy, like nitinol, and may be designed to have a particular shape and/or geometry. Through constant or intermittent contact with the inner wall of the blood vessel or some other anatomical feature, struts can provide localization or positioning or both. A device to be positioned may have multiple sets of struts and these may project from the device at one or more angles or at any angle. In some embodiments, the struts may have features like hooks. In other embodiments, the struts may have pads to interface with the surface of the blood vessel wall. For use with intravascular devices, struts may have a collapsed configuration for fitting within a sheath and an expanded configuration to provide localization and/or positioning. In some embodiments the struts may have knees (or kinks or bends) to prevent hooks or other features from contacting the inner wall of a sheath in a collapsed configuration. The struts may be shaped, formed, and processed so that for a given outward radial force in the expanded configuration, the radial force in the collapsed configuration and/or the force required to move from the expanded to collapsed configuration is reduced or minimized.

In some embodiments, the localization, stabilization, and positioning system (or support structure) may comprise one or more tethers that connect the device to be localized and/or positioned to one or more anchor or contact points. Tethers may be flexible and may preferentially limit translation or rotation in one direction. In some embodiments, the tethers may have an additional function. As one nonlimiting example, a tether may also comprise a power lead that transmits electrical power to the device to be localized or positioned.

In some embodiments, the localization, stabilization, and positioning system may comprise a means of propulsion (e.g., a pump in various embodiments). In cases where the device to be localized and/or positioned is an intravascular blood pump, the pumping of blood is a key function of the device in some embodiments. The propulsive or reactive force generated by blood pumping may be used as part of the localization and positioning system.

In some embodiments, the localization and/or positioning system may comprise combinations of the above elements that together provide unique benefits or advantages.

The discussion herein has outlined rather broadly various features of the present disclosure in order that the detailed description that follows may be better understood. Additional features and advantages of the disclosure will be described hereinafter.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1A is a schematic perspective, partially-exploded view of a blood flow assist system, according to various embodiments.

FIG. 1B is a schematic perspective view of a pump at a distal portion of the blood flow assist system of FIG. 1A.

FIG. 1C is a schematic perspective, partially-exploded view of the pump of FIG. 1B.

FIG. 1D is a schematic side sectional view of a motor housing according to various embodiments.

FIG. 1E is a schematic perspective view of a motor and a motor mount support.

FIG. 1F is a schematic perspective view of a distal end of a power lead having lumens shaped to received conductors that are configured to supply power to the motor.

FIG. 1G is a schematic perspective view of a proximal end portion of the power lead.

FIG. 1H is a schematic side view of the pump disposed in a collapsed configuration in a delivery sheath.

FIG. 1I is a schematic perspective view of a retrieval feature used to remove the pump, according to some embodiments.

FIG. 1J is a cross-sectional view of an alternative embodiment in which a drive shaft is coupled to a motor configured to be disposed outside the patient when the pump is in use.

FIG. 2 is a schematic exploded view of a pump according to various embodiments and steps in a method of assembling the pump.

FIGS. 2A-2B schematically illustrate an example of a drive module, according to various embodiments.

FIGS. 2C-2I illustrate example components of a hydraulic module, according to various embodiments.

FIGS. 3A-3C illustrate embodiments of a power module for supplying power to drive modules such as those of FIGS. 2-2B.

### DETAILED DESCRIPTION

Refer now to the drawings wherein depicted elements are not necessarily shown to scale and wherein like or similar elements are designated by the same reference numeral through the several views.

Referring to the drawings in general, it will be understood that the illustrations are for the purpose of describing particular implementations of the disclosure and are not intended to be limiting thereto. While most of the terms used herein will be recognizable to those of ordinary skill in the art, it should be understood that when not explicitly defined, terms should be interpreted as adopting a meaning presently accepted by those of ordinary skill in the art.

### I. OVERVIEW OF BLOOD FLOW ASSIST SYSTEMS

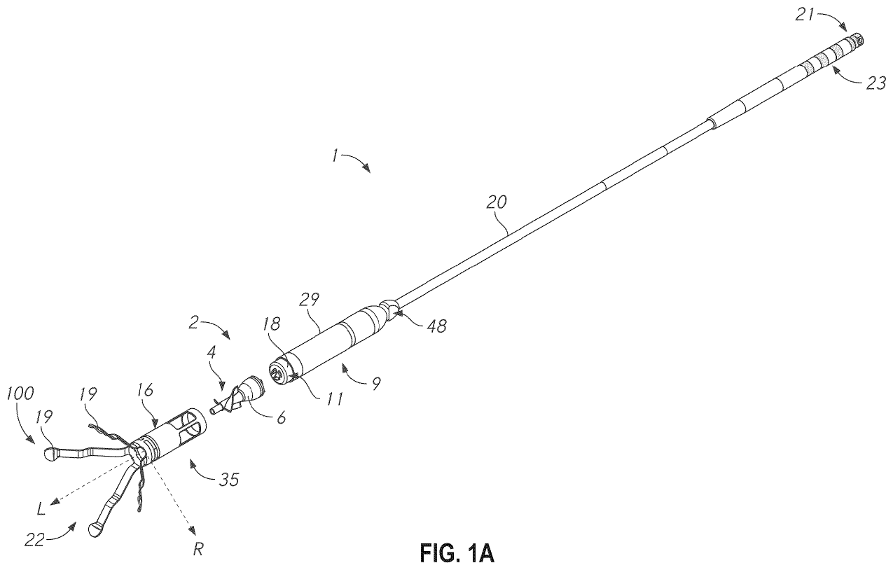

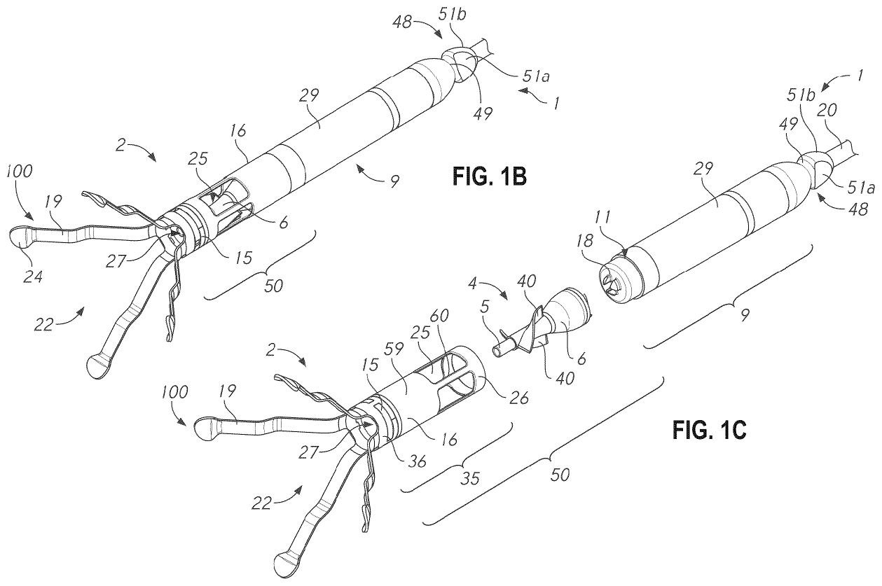

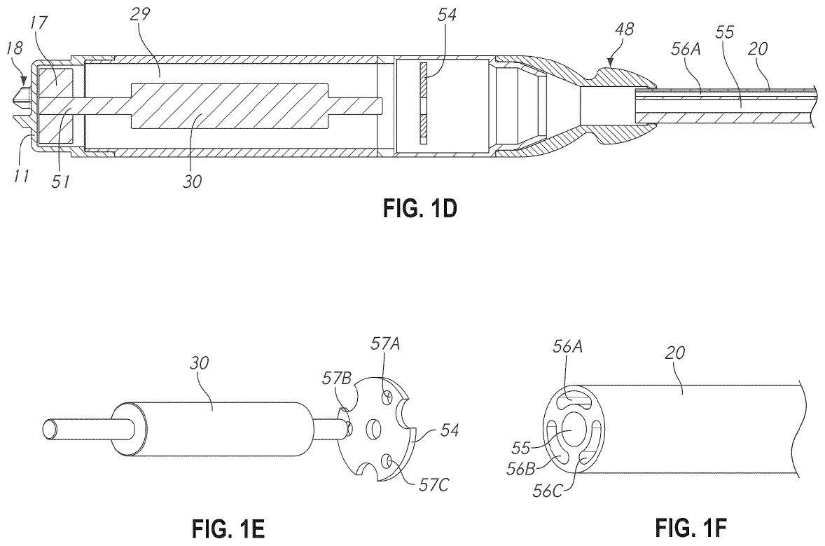

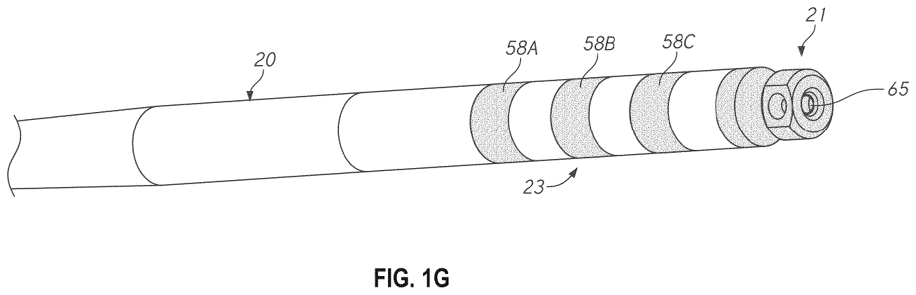

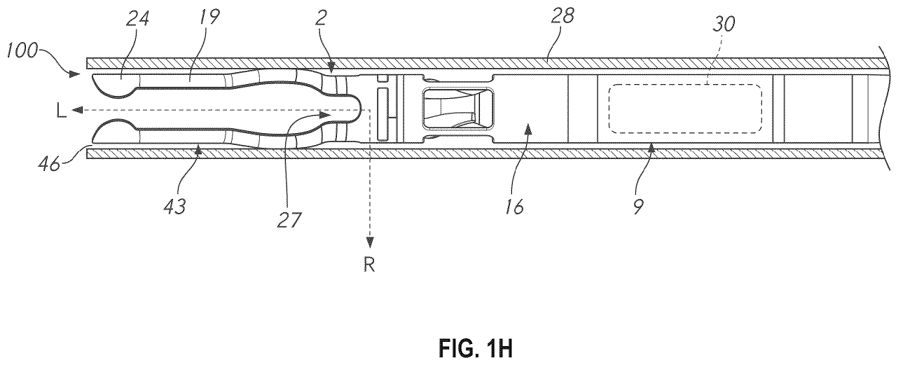

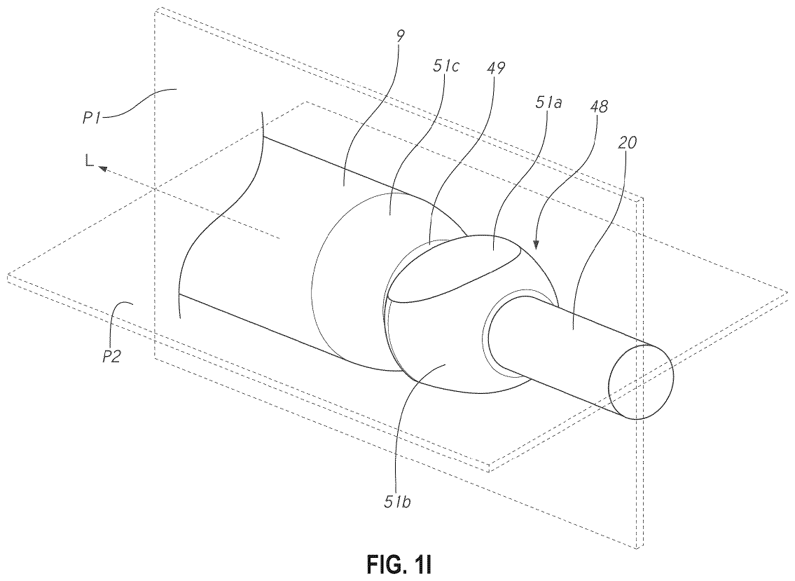

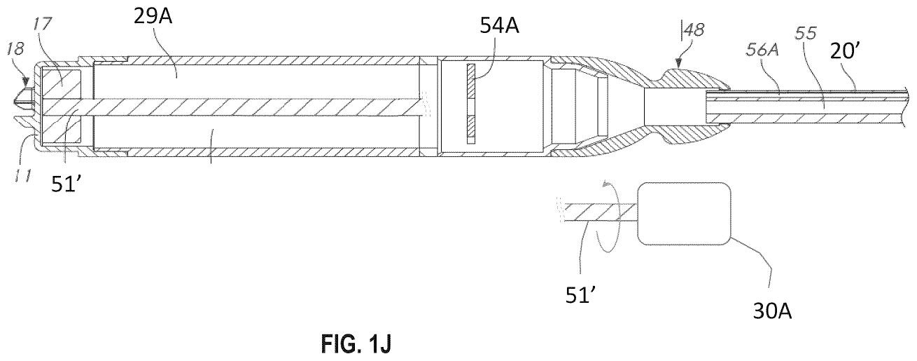

Various embodiments disclosed herein relate to a blood flow assist system 1 configured to provide circulatory support to a patient, as illustrated in FIGS. 1A-1I. Section II below discussed various advantageous methods of assembling components of the blood flow assist system 1. The system 1 can be sized for intravascular delivery to a treatment location within the circulatory system of the patient, e.g., to a location within the descending aorta of the patient. As shown in FIG. 1A, the system 1 can have a proximal end 21 with a connector 23 configured to connect to an external control system, e.g., a console (not shown). The connector 23 can provide electrical communication between the control system and a power lead 20 extending distally along a longitudinal axis L from the connector 23 and the proximal end 21. The power lead 20 can comprise an elongate body that electrically and mechanically connects to a pump 2 at or near a distal end 22 of the blood flow assist system 1, with the distal end 22 spaced apart from the proximal end 21 along the longitudinal axis L. As explained herein, the power lead 20 can also serve as a flexible tether configured to oppose loads applied in opposite directions at opposite ends of the power lead 20.

The pump 2 can comprise a pump head 50 including a pump housing 35 connected to a drive unit 9 that includes a motor housing 29. A retrieval feature 48 can be provided at a proximal end portion of the pump 2. In some embodiments, the retrieval feature can be coupled with the distal end of the power lead 20 between the power lead 20 and the motor housing 29. After a procedure, the clinician can remove the pump 2 from the patient by engaging a tool (e.g., a snare, a clamp, hook, etc.) with the retrieval feature 48 to pull the pump 2 from the patient. For example, the retrieval feature 48 can comprise a neck 49 (e.g., a reduced diameter section) at a proximal curved portion 51c of the motor housing 29 and an enlarged diameter section disposed proximal the neck 49. The enlarged diameter section can comprise a first curved portion 51a and a second curved portion 51b, as shown in FIGS. 1B, 1C, and 1I. The first and second curved portions 51a, 51b can comprise convex surfaces, e.g., convex ball portions. The first and second curved portions 51a, 51b can have different radii of curvature. For example, as shown in FIG. 1I, the first curved portion 51a can have a larger radius of curvature than the second curved portion 51b. The first curved portion 51a can be disposed on opposing sides of the retrieval feature 48 in some embodiments. The second curved portion 51b can be disposed around the first curved portion 51a and can have a radially-outward facing surface and a proximally-facing convex surface coupled to the distal end of the power lead 20. The neck 49 can have a first depth at a first circumferential position of the retrieval feature 48 and a second depth less than the first depth at a second circumferential position of the retrieval feature 48 spaced apart from the first circumferential position.

Beneficially, as shown in FIG. 1I, one or more first planes P1 extending parallel to the longitudinal axis L and intersecting the first curved portion 51a can have a first angle or taper between the proximal curved portion 51c of the motor housing 29 and the first curved portion 51a. One or more second planes P2 extending parallel to the longitudinal axis L and intersecting the second curved portion 51b can have a second angle or taper (which is different from the first angle or taper) between the proximal curved portion 51c of the motor housing 29 and the second curved portion 51b. The first angle or taper can provide a gradual, continuous (generally monotonically decreasing) geometric transition between the proximal curved portion 51c of the motor housing 29 and the power lead 20, which can provide for smooth blood flow and reduce the risk of thrombosis. The second curved portion 51b can serve as a lobe that extends radially outward, e.g., radially farther out than the first curved portion 51a. The second curved portion 51b can be used to engage with a retrieval device or snare to remove the pump 2 from the anatomy. Some cross sections through the longitudinal axis of the retrieval feature 48 can contain a substantial neck (e.g., a local minimum in the radius of curvature measured along its central axis) while other cross sections through the longitudinal axis of the retrieval feature 48 can contain an insubstantial local minimum or no local minimum. In the illustrated embodiment, there are two first curved portions 51a that can serve as a dual lobe retrieval feature. In other embodiments, more or fewer lobes can be provided to enable pump retrieval while ensuring smooth flow transitions between the motor housing 29 and power lead 20.

As shown in FIGS. 1B-1C, 1E, and 1I, the neck 49 can be disposed between the curved portions 51a, 51b and a proximally-facing convex surface 51c of the motor housing 29. In the illustrated embodiment, the retrieval feature 48 can be coupled to or integrally formed with the motor housing 29. In other arrangements, the retrieval feature 48 can be disposed at other locations of the pump 2. As shown, the retrieval feature 48 can be symmetrical and continuously disposed about the longitudinal axis L. In other arrangements, the retrieval feature 48 can comprise a plurality of discrete surfaces spaced apart circumferentially and/or longitudinally. In the illustrated embodiments, the motor housing 29 (and motor) can be part of the pump 2 and disposed inside the vasculature of the patient in use. In other embodiments, however, the motor housing 29 (and motor) can be disposed outside the patient and a drive cable can connect to the impeller 6.

As shown in FIGS. 1A-1C, the drive unit 9 can be configured to impart rotation to an impeller assembly 4 disposed in the pump housing 35 of the pump head 50. As explained herein, the drive unit 9 can include a drive magnet 17 (see FIG. 1D) and a motor 30 (see FIGS. 1D-1E) disposed in the motor housing 29 capped by a distal drive unit cover 11. The motor 30 is shown schematically in FIG. 1D. The drive unit cover 11 can be formed with or coupled to a drive bearing 18. The drive magnet 17 can magnetically couple with a corresponding driven or rotor magnet (not shown) of the impeller assembly 4 that is disposed proximal the impeller 6 within the shroud 16. The power lead 20 can extend from the treatment location to outside the body of the patient, and can provide electrical power (e.g., electrical current) and/or control to the motor 30. Accordingly, no spinning drive shaft extends outside the body of the patient in some embodiments. As explained herein, the power lead 20 can energize the motor 30, which can cause the drive magnet 17 to rotate about the longitudinal axis L, which can serve as or be aligned with or correspond to an axis of rotation. Rotation of the drive magnet 17 can impart rotation of the rotor magnet and a primary or first impeller 6 of the impeller assembly 4 about the longitudinal axis L. For example, as explained herein, the rotor magnet (which can be mechanically secure to an impeller shaft 5) can cause the impeller shaft 5 (which can serve as a flow tube) and the first impeller 6 to rotate to pump blood. In other embodiments, the drive unit 9 can comprise a stator or other stationary magnetic device. The stator or other magnetic device can be energized, e.g., with alternating current, to impart rotation to the rotor magnet. In the illustrated embodiments, the impeller 6 can have one or a plurality of blades 40 extending radially outward along a radial axis R that is radially transverse to the longitudinal axis L. For example, the first impeller 6 can have a plurality of (e.g., two) longitudinally-aligned blades 40 that extend radially outwardly from a common hub and that have a common length along the longitudinal axis L. The curvature and/or overall profile can be selected so as to improve flow rate and reduce shear stresses. Skilled artisans would appreciate that other designs for the first impeller 5 may be suitable.

As shown in FIGS. 1A-1C, the impeller assembly 4 can be disposed in a shroud 16. The impeller shaft 5 can be supported at a distal end by a sleeve bearing 15 connected to a distal portion of the shroud 16. A support structure such as a localization system 100 (discussed further below) can comprise a base portion 36 coupled with the sleeve bearing 15 and/or the shroud 16. In some embodiments, the base portion 36, the sleeve bearing 15, and/or the shroud 16 can be welded together. In other embodiments, the sleeve bearing 15 and/or the shroud 16 can be formed as one part. The base portion 36 of the support structure or localization system 100 (which can be part of or serve as a support structure), the sleeve bearing 15, and the shroud 16 can cooperate to at least partially define the pump housing 35, as shown in FIGS. 1A and 1C. The localization system 100 can comprise a plurality of self-expanding struts 19 having convex contact pads 24 configured to contact a blood vessel wall to maintain spacing of the pump housing 35 from the wall of the blood vessel in which the pump housing 35 is disposed. In FIGS. 1A-1C, the struts 19 of the localization system 100 are illustrated in an expanded, deployed configuration, in which the contact pads 24 extend radially outward to a position in which the contact pads 24 would contact a wall of a blood vessel within which the pump 2 is disposed to at least partially control position and/or orientation of the pump head 50 relative to the blood vessel wall, e.g., to anchor, the pump 2 during operation of the system 1.

A first fluid port 27 can be provided distal the impeller assembly 4 at a distal end of the pump housing 35. The shroud 16 can comprise a proximal ring 26 coupled with the motor housing 29 and a plurality of second fluid ports 25 formed in a proximal portion of the shroud 16 adjacent (e.g., immediately distal) the proximal ring 26. As shown in FIG. 1C, the second fluid ports 25 can comprise openings formed between axially-extending members 60 (also referred to as pillars) that extend along the longitudinal axis L (which may also serve as a longitudinal axis of the pump head 2 and/or pump housing 35) between the proximal ring 26 and a cylindrical section 59 of the shroud 16. In some embodiments, the axially-extending members 60 can be shaped or otherwise be configured to serve as vanes that can shape or direct the flow of blood through the second fluid ports 25. For example, in various embodiments, the axially-extending members 60 can be angled, tapered, or curved (e.g., in a helical pattern) to match the profile of the impeller blades 40 and/or to accelerate blood flow through the pump 2. In other embodiments, the axially-extending members 60 may not be angled to match the blades 40. In some embodiments, the first fluid port 27 can comprise an inlet port into which blood flows. In such embodiments, the impeller assembly 4 can draw blood into the first fluid port 27 and can expel the blood out of the pump 2 through the second fluid ports 25, which can serve as outlet ports. In other embodiments, however, the direction of blood flow may be reversed, in which case the second fluid ports 25 may serve as fluid inlets and the first fluid port 27 may serve as a fluid outlet.

As shown in FIGS. 1A-1D, the system 1 comprises the drive unit 9 with the motor 30 that can be sealed in the motor housing 29. The drive magnet 17 can be rotatable by the motor 30 by way of a motor shaft 51. The motor 30 can electrically connect to the power lead 20. The power lead 20 can serve as a flexible tether that comprises an elongate tension member configured to oppose loads applied in opposite directs at opposite ends of the power lead 20. In one embodiment the power lead 20 is hollow, as discussed further below. As shown in FIGS. 1D and 1F, the power lead 20 can comprise an insulating body having a central lumen 55 and a plurality of (e.g., three) outer lumens 56A-56C extending along a length of the power lead 20. One or more electrical conductors can be disposed in the hollow elongate power lead 20 and can be configured to convey current to the motor 30 from a source, such as the external control system. For example, in some embodiments, the outer lumens 56A-56C can be sized and shaped to receive corresponding electrodes or electrical wires (not shown) to provide electrical power to the motor 30. For example, the lumens 56A-56C can receive, wires configured to supply ground and drive voltage to corresponding windings on the motor. The electrodes can extend through corresponding openings 57A-57C of a motor mounting support 54 configured to support the motor 30. The central lumen 55 can be sized and shaped to receive an elongate stiffening member or guidewire (not shown). The stiffening member or guidewire can be inserted through an opening 65 at the proximal end 21 (see FIG. 1G) into the central lumen 55 during delivery to help guide the pump 2 to the treatment location or maintain the pump 2 in a given location. The stiffening member or guidewire can be easily inserted and removed when finished. As shown in FIG. 1G, the connector 23 near the proximal end 21 of the system 1 can have electrical contacts 58A-58C electrically connected to the wires or conductors in the corresponding outer lumens 56A-56C. The contacts 58A-58C can comprise rings spaced apart by an insulating material and can be configured to electrically connect to corresponding electrical components in the control system or console (not shown).

Beneficially, the blood flow assist system 1 can be delivered percutaneously to a treatment location in the patient. FIG. 1H shows the pump 2 disposed within an elongate sheath 28. As shown, the struts 19 are held in a collapsed configuration by the inner wall of the sheath 28. As discussed further below, the struts 19 can be configured to collapse in a controlled manner, e.g., with at least a portion deflected away from inner wall of the sheath 28 when disposed in the sheath. As shown, the struts 19 can comprise knees 102, which can serve to space distal ends of the struts 19 (e.g., at or near the contact pads 24 or hooks) from the inner wall of the sheath 28, such that there is a space 46 between the contact pads 24 or hooks and the inner wall of the sheath 28 in the collapsed configuration within the sheath 28.

The knees 102 can be of the same configuration for each of the struts 19 in one embodiment. In such an embodiment, the struts 19 may all collapse or fold in the same manner within the sheath 28. In another embodiment the knee 102 of one or more struts 19 can be differentiated from the knee 102 of one or more other struts 19 such that the struts are collapsed or folded in different manners. As explained herein, in various embodiments, the struts can be longitudinally-aligned or longitudinally-offset or staggered. For example, a pair of opposing struts 19 (e.g., disposed radially opposite one another) can have knees 102 that cause the opposing strut of the pair to collapse prior to the collapsing of other struts 19 of the pump 2. In one example, the pump 2 has four struts 19. Two opposing struts 19 are configured to bend at the knees 102 prior to the bending of the knees of the other struts 19. As such, the two opposing struts 19 can be collapsed to a position between the other two struts to provide a compact arrangement. The knees 102 can be configured such that some struts undergo a greater degree of bending or collapsing. Thus the space 46 between the contact pads 26 and the inner wall of the sheath 28 can be two to six (and in some cases three to four) times greater for one or more, e.g., a pair of, struts than for one or more, e.g., another pair of struts 19, which can be provided to avoid tangling of the struts. Accordingly, in various embodiments, some struts may be structured to collapse first when engaged with the sheath 28, and the remaining struts can collapse as the sheath 28 induces the collapsing of the initial struts.

In some embodiments, one or more struts comprises knees 102 that can control the order of collapsing of the struts. For example one or more struts can have a knee 102 positioned more proximally compared to the position of the knees 102 of one or more other struts. In one example, two opposing struts 19 can have knees 102 disposed more proximally than are the knees 102 of another strut 19. In one example, a first set of opposing struts 19 have knees 102 disposed more proximally than a second set of struts 19 disposed approximately 90 degrees offset from the first set of struts 19. This can allow the first set of struts to be more completely folded by distal advancement of the sheath 28 before a more complete folding of the second set of struts 19. In a further variation, knees 102 can be longitudinally spaced apart on adjacent struts 19 so that adjacent struts fold at different times or rates. The illustrated embodiments includes the knees 102, but in other embodiments, no knees may be provided. For example, the struts 19 can be retracted at different rates by hinges and/or by modifying material thickness or properties in or along the length of one or more struts 19 to control the timing or rate of folding upon advancing the sheath 28. A living hinge structure can be formed along the length of one or more struts 19 to control timing, rate, and/or sequence of retraction of the struts 19. In one example, an area of reduced thickness transverse to the length of a strut 19 causes the strut to fold or bend when a sheath is advanced across the reduced thickness area. By offsetting the longitudinal position of reduced thickness areas in the struts 19, the sequence of retraction can be controlled.

In the collapsed configuration, the struts 19 can be compressed to a diameter or major lateral dimension at one or more locations that is approximately the same as (or slightly smaller than) the diameter of the shroud 16. Thus, as shown in the collapsed configuration of FIG. 1H, at least a portion of the struts 19 are compressed to a diameter or major lateral dimension that is smaller than the major lateral dimension or diameter of the pump housing 35, shroud 16 and/or the drive unit 9. In some embodiments, at least a portion of the struts has a major lateral dimension that is no more than a major lateral dimension of the pump housing 35. In some embodiments, at least a portion of the struts has a major lateral dimension that is less than a major lateral dimension of the pump housing 35 and/or the motor housing 29. The patient can be prepared for the procedure in a catheterization lab in a standard fashion, and the femoral artery can be accessed percutaneously or by a surgical approach. The sheath 28 (or a dilator structure within the sheath 28) can be passed over a guidewire and placed into the treatment location, for example, in the descending aorta. After the sheath 28 is placed (and the dilator removed), the pump 2 can be advanced into the sheath 28, with the pump 2 disposed in the mid-thoracic aorta, approximately 4 cm below the take-off of the left subclavian artery. In other embodiments, the pump 2 and sheath 28 can be advanced together to the treatment location. Positioning the pump 2 at this location can beneficially enable sufficient cardiac support as well as increased perfusion of other organs such as the kidneys. Once at the treatment location, relative motion can be provided between the sheath 28 and the pump 2 (e.g., the sheath 28 can be retracted relative to the pump 2, or the pump 2 can be advanced out of the sheath 28). The struts 19 of the localization system can self-expand radially outwardly along the radial axis R due to stored strain energy into the deployed and expanded configuration shown in FIGS. 1A-1C. In some embodiments, such as those in which the vasculature is accessed by the femoral artery, the struts 19 can extend distally, e.g., distally beyond a distal end of the shroud 16 and/or the impeller 6. In other embodiments, as explained herein, the pump 2 can be delivered percutaneously through a subclavian artery. In such embodiments, the struts 19 may extend proximally, e.g., proximal the pump housing 35 and/or the motor housing 29. In still other embodiments, multiple pluralities of struts may extend proximally and distally relative to the pump 2. The convex contact pads 24 can engage the blood vessel wall to stabilize (e.g., assist in anchoring) the pump 2 in the patient's vascular system. Once at the treatment location, the clinician can engage the control system to activate the motor 30 to rotate the impeller assembly 4 to pump blood.

Thus, in some embodiments, the pump 2 can be inserted into the femoral artery and advanced to the desired treatment location in the descending aorta. In such arrangements, the pump 2 can be positioned such that the distal end 22 is upstream of the impeller 6, e.g., such that the distally-located first fluid port 27 is upstream of the second fluid port(s) 25. In embodiments that access the treatment location surgically or percutaneously via the femoral artery, for example, the first fluid port 27 can serve as the inlet to the pump 2, and the second ports 25 can serve as the outlet(s) of the pump 2. The struts 19 can extend distally beyond a distal end of the pump housing 35. In other embodiments, however, the pump 2 can be inserted percutaneously through the left subclavian artery and advanced to the desired treatment location in the descending aorta. In such arrangements, the pump 2 can be positioned such that the distal end 22 of the system 1 is downstream of the impeller 6, e.g., such that the distally-located first fluid port 27 is downstream of the second fluid port(s) 25. In embodiments that access the treatment location through the left subclavian artery, the second fluid port(s) 25 can serve as the inlet(s) to the pump 2, and the first port 27 can serve as the outlet of the pump 2.

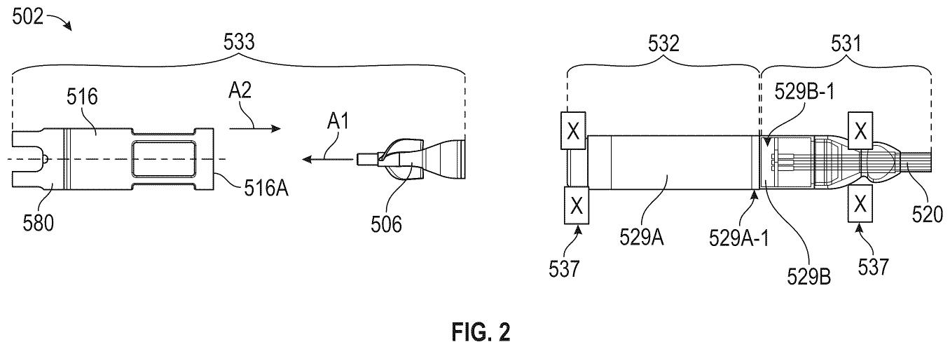

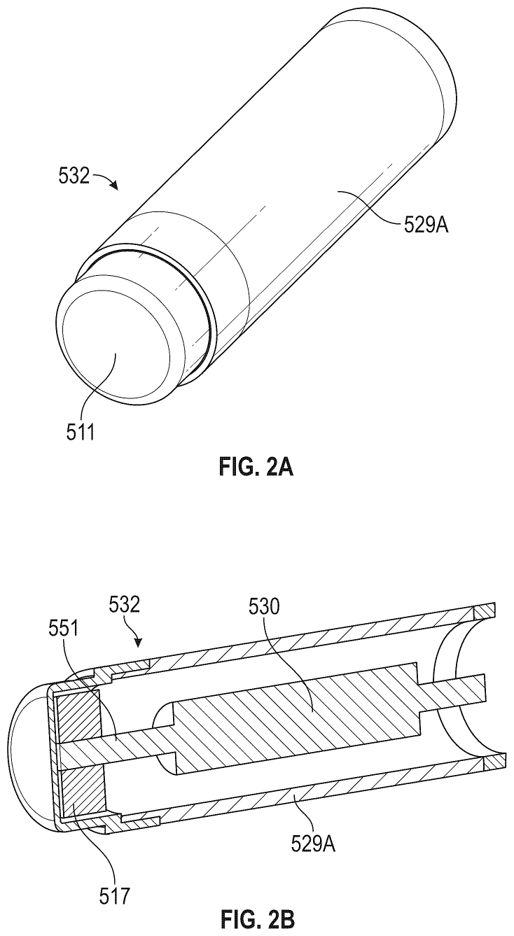

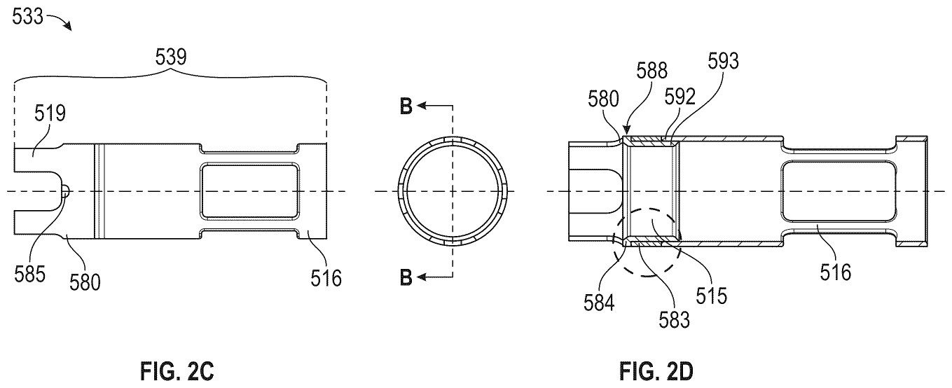

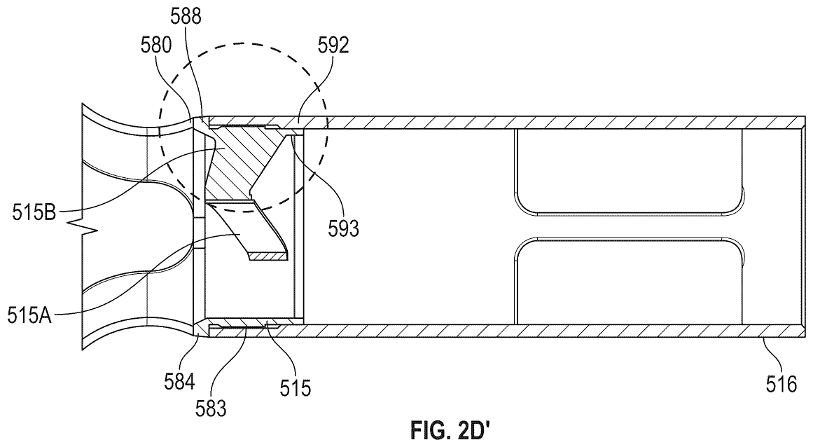

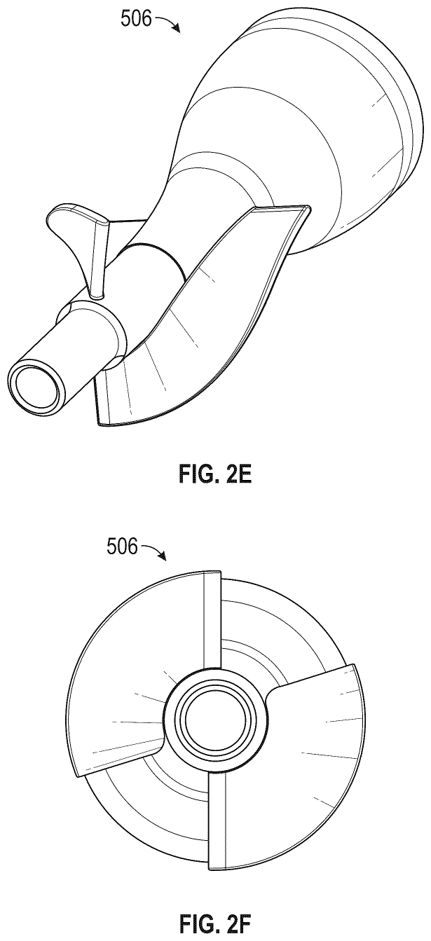

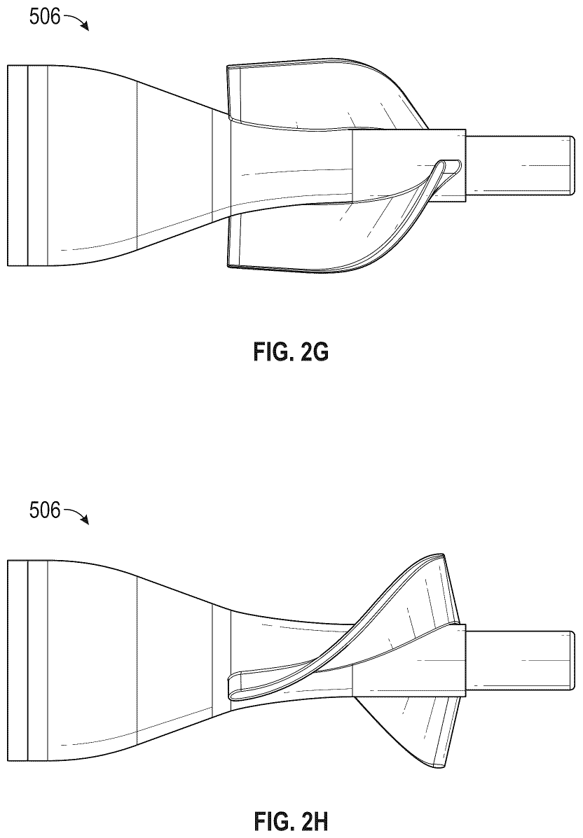

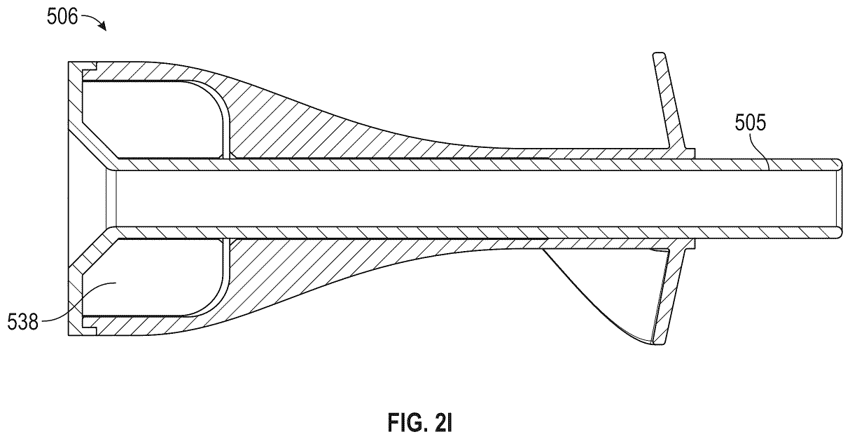

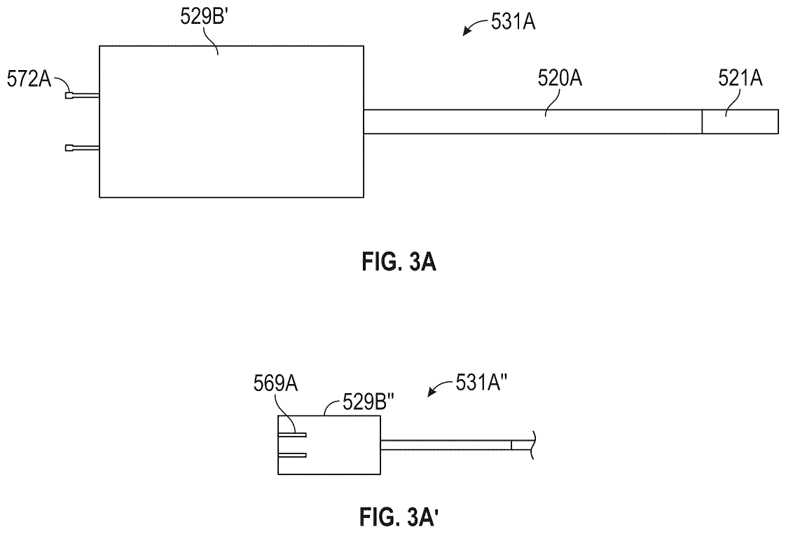

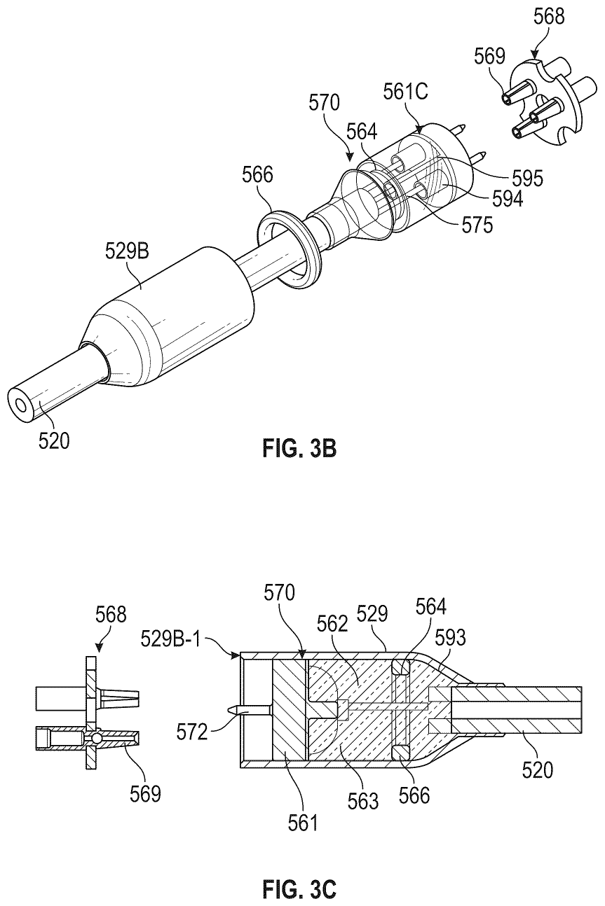

When the treatment procedure is complete, the pump 2 can be removed from the patient. For example, in some embodiments, the pump can be withdrawn proximally (and/or the sheath 28 can be advanced distally) such that a distal edge of the sheath 28 engages with a radially-outer facing surface 43 of the struts 19. In some embodiments, the distal edge of the sheath 28 can engage with the knees 102 of the struts (see, e.g., FIGS. 2A-3C). The distal edge of the sheath 28 can impart radially-inward forces to the radially-outer facing surface 43 (e.g., at approximately the location of the knees 102) to cause the struts 19 to collapse and be drawn inside the sheath 28. Relative motion opposite to that used for deploying the pump 2 can be provided between the sheath 28 and the pump 2 (e.g., between the sheath 28 and the impeller assembly 4 and pump housing 35) to collapse the struts 19 into the sheath 28 in the collapsed configuration. In some embodiments, the pump 2 can be withdrawn from the sheath 28 with the sheath 28 in the patient's body, and the sheath 28 can be subsequently used for another procedure or removed. In other embodiments, the sheath 28 and the pump 2 can be removed together from the patient's body.

The foregoing description includes embodiments in which a proximal end of a drive shaft 51 is located in the drive unit 9. The proximal end of the drive shaft 51 and the motor 30 are disposed within the body in use. FIG. 1J shows another embodiment in which a motor 30A is disposed outside the body in use. An elongate, flexible shaft 51′ is coupled at a distal end with the drive magnet 17. The shaft 51′ extends through an elongate body 20′ and is or can be coupled at a proximal end thereof with a motor 30A. The motor 30A can be larger than the motor 30 since it need not be disposed within the profile of the sheath 28. The elongate body 20′ may have one or more lumens. The shaft 51′ may extend through the central lumen 55. One or more outer lumens 56a may be provided to flow a fluid into the system to lubricate and/or cool the shaft 51′. Rotation of the proximal end of the shaft 51′ by the motor 30a results in rotation of the entire length of the shaft 51′ through the elongate body 20′ and also results in rotation of the drive magnet 17. Rotation of the drive magnet 17 causes rotation of one or more magnets in the impeller 6 to create flow through the pump 2 by virtue of magnetic attraction of these magnets across the distal drive unit cover. In other embodiments, the shaft 51′ can be directly mechanically coupled to the impeller 6 such that rotation does not depend on magnetic coupling. One or more shaft rotation supports 54A can be provided within a distal housing 29A to support a distal portion of the shaft 51′. The elongate body 20′ and/or the shaft 51′ can comprise a tether to control or to aid in control of the position of the pump, e.g., to counter thrust forces of the impeller 6 to reduce or minimize movement of the pump 2 in operation.

Additional details of the pump 2 and related components shown in FIGS. 1A-1H may be found throughout International Patent Application No. PCT/US2020/062928, filed on Dec. 2, 2020, the entire contents of which are incorporated by reference herein in their entirety and for all purposes. Prior work is also detailed in U.S. Pat. No. 8,012,079 and U.S. Pat. Pub. No. 2017/0087288, which are both fully incorporated by reference herein.

### II. EXAMPLES OF METHODS FOR MANUFACTURING MODULAR MEDICAL DEVICES

Various embodiments disclosed herein relate to the assembly of modular medical devices, such as percutaneous cardiac assist devices (e.g., an electromotive micromechanical heart pump). As explained herein, a percutaneous cardiac assist device 500 can comprise a fully implantable heart pump including a power delivery module 531 (e.g., a power delivery section), a drive module 532 (e.g., an electromotive power section or other device energized by electrical power supplied by the power delivery section to perform work) and a hydraulic module 533 (e.g., a pump head and impeller section or other output device performing work). Because the various modules or sections can be made from different materials and may use section-specific manufacturing techniques, modularizing the construction and assembly of the pump has benefits for manufacturing, quality, and performance considerations. It should be appreciated that although the examples disclosed herein relate to percutaneous cardiac assist devices, in various embodiments, the medical device can comprise any suitable type of device, e.g., any suitable type of percutaneous or fully implantable medical device. The devices, systems, and methods disclosed herein can be utilized in combination with any of the devices, systems, and methods disclosed in U.S. patent application Ser. No. ______, having attorney docket number PRCY.029A, filed Nov. 7, 2025, entitled “MODULAR STRUT ASSEMBLY FOR INTRAVASCULAR DEVICES,” and in U.S. patent application Ser. No. ______, having attorney docket number PRCY. 030A, filed Nov. 7, 2025, entitled “HERMETIC DRIVELINE FOR IMPLANTABLE MEDICAL DEVICES,” the entire contents of each of which are hereby incorporated by reference herein in their entirety and for all purposes.

One example of such a pump includes a modular electromechanical drive unit (drive module 532) attached to a modular electric power delivery section (power module 531) at a first end portion (e.g., a proximal end portion) of the drive module 532 and attached to the pump head/impeller section (hydraulic module 533) at a second end portion (e.g., a distal end portion) of the drive module 532. FIG. 2 is a schematic exploded view of a pump 502 according to various embodiments. Unless otherwise noted, the components of FIG. 2 can be generally similar to or the same as like numbered components in FIGS. 1A-1J, with the reference numerals having “5” as a prefix relative to the reference numerals of FIGS. 1A-1J. The materials used in the modules, the methods of construction, assembly environment, and design of each module can be independent, permitting and enabling modularization of the process of pump assembly. For example, an axial flow heart pump sized to fit within the human vasculature can use the principle of entrainment to add momentum to the native blood flow at a desired site. Examples of such heart pumps may be found throughout U.S. Pat. Nos. 10,500,323; 12,078,192; 11,324,940; and 11,471,665, the entire contents of which are incorporated by reference in their entirety and for all purposes. The hydraulic module 533 can be magnetically and mechanically coupled to the drive module 532, and the power module 531 can be mechanically and electrically coupled to the drive module 532.

The micromechanical pump 502 can be assembled from the constitutive modules, e.g., the power module 531, the drive module 532, and the hydraulic module 533. FIG. 2 also illustrates a step of a method of assembling the modules 531, 532, 533 as discussed further below. An impeller 506 is inserted into a shroud 516 as indicated by arrow A1. This step can form one variation of the hydraulic module 533. After the power module 531 has been joined to the drive module 532, the hydraulic module 533 can be positioned adjacent to the drive module 532, as indicated by arrow A2, prior to securing the shroud 516 to the motor housing 529A. In one embodiment the modules can be mechanically connected by welding, e.g., by circumferential laser welding. For example, as shown, the power module 531 can be fitted to the drive module 532 such that male or female electrical pins on the power module 531 fit into corresponding male or female pins on the drive module 532. Once fitted, the corresponding housings 529A, 529B of the drive module 532 and the power module 531 will be in apposition and aligned.

The mated drive module 532 and power module 531 can be placed into one or more jigs 537 which maintains alignment of (e.g., a position and/or an orientation of) the two modules 531, 532 while permitting rotation of the two aligned modules 531, 532. The jig 537 with the modules 531, 532 can then be placed into a laser welder in which the rotatory portion of the jig 537 can be attached to the collet or drive mechanism of the welder. The laser welder can mechanically connect the respective housings 529A, 529B of the drive module 532 and the power module 531 by welding them together while the jig 537 is axially rotated during application of laser energy. At the completion of this assembly step, the drive module 532 can be hermetically sealed and the power module 531 can be ionically sealed against intrusion. In another technique the jigs 537 and the modules 531, 532 can be disposed in a rotating welding apparatus to form a circumferential weld. In another apparatus, but the jigs 537 and the modules 531, 532 as well as the welding apparatus can be rotated to form a circumferential weld.

The assembly process can continue by positioning the mated and connected drive and power units 531, 532 in a similar rotatory jig or jigs which also aligns the hydraulic module 533 so that the hydraulic module 533 can be mechanically attached by laser welding to the drive and power unit assembly (e.g., the mated power and drive modules 531, 532). Accordingly, the micromechanical pump 602 can be assembled by these steps in a modular manner which can simplify manufacturing.

FIGS. 2A-2B schematically illustrate an example of the drive module 532. In various embodiments, the drive module 532 can include various electromechanical components, including a motor shaft 551, motor windings, and magnets (e.g., a drive magnet 517 connected to motor shaft 551), encapsulated in a cylindrical shell or first housing 529A (also referred to herein as a motor housing). At one end of the motor shaft the drive magnet 517 is affixed, which is in turn capped (e.g., by drive unit cover 511) such that the end is hermetically sealed. The end of the shell or first housing 529A opposite to the drive unit cover 511 contains or includes mechanical and electrical connections to receive the power delivery module 531.

FIGS. 2C-2I illustrate example components of the hydraulic module 533, including a strut assembly 580 connected to a distal bearing 515 (which may be similar to the sleeve bearing 15 in some embodiments) and shroud 516. The strut assembly 580 is also sometimes referred to as a strut module and may be formed of a monolithic construction. The impeller 506 can be disposed within the shroud 516 as explained herein. The hydraulic module 533 can include the shroud 516 or nacelle (e.g., as used herein, a nacelle can be a tubular enclosure for an impeller) which houses the impeller 506. In addition, the strut assembly or strut module 580 including a plurality of struts 519 or tines can be attached to the shroud 516 so as to provide localization (e.g., centralization and stabilization) of the assembled pump 502 when implanted. The struts 519 can comprise any of the struts disclosed herein. In one embodiment the tines or struts 519 can be made from a shape memory alloy (e.g., nitinol) so that the tines or struts 519 may be compressed while passing through the vasculature and subsequently self-expand to seat and centralize the pump 502.

To facilitate the modularization of the hydraulic module 533, the shroud 516, which in some embodiments may also contain a bearing, can be mated or connected to the strut assembly 580 (including the struts 519 or tines). Welding is one method for connecting the components of the hydraulic module 533 together. However, some shape memory alloys may not be amenable to welding to lower melt temperature alloys such as titanium alloys and therefore a compression capturing mechanism wherein a through weld is used between like materials can facilitate modularization of the hydraulic module 533. The compression capturing method can include slidably mechanically coupling a bearing ring 588 with a proximal ring 583 of the strut assembly or strut module 580. The slidably coupling can be followed by compression of this assembly with the shroud 516 prior to a single welding step forming one or a plurality of weld locations, e.g., one, two, four, eight, more than two, more than four, or more than 8 weld locations.

In various embodiments, for example, the strut module 580 can be connected to the shroud 516 by a single welding process, eliminating the need for additional welding or adhesives. The module can be assembled by sliding a bearing ring 588 of the distal bearing 515 within a proximal ring 583 of the strut module 580. A first locating feature 584 of the bearing ring 588 can be aligned with a second locating feature 585 of the strut module 580. In the illustrated embodiment, the first locating feature 584 can comprise a radial projection and the second locating feature 585 can comprise a recess extending from an outside surface of the proximal ring 583 to an inside surface of the proximal ring 583. As shown the radial projection of the first locating feature 584 can extend radially outward from a distal edge of the proximal ring 583. In the illustrated embodiment, the shroud 516 can be welded to the bearing ring 588 by a single welding step. The single welding step can be performed at a plurality of locations about the circumference of the shroud 516.

Although a bearing 515 is shown in FIGS. 2C-2D, in other embodiments, the separate bearing 515 may be omitted. In some embodiments, the strut module 580 and shroud 516 can be formed of a unitary component. Accordingly, in various embodiments, the hydraulic module can comprise a collar 539 disposed about a rotary or movable device, such as the impeller 506. In the illustrated embodiment, the collar 539 can comprise the strut module 580, the bearing 515, and the shroud 516, but in other embodiments the collar 539 can comprise any other suitable structure (e.g., a unitary structure, or a structure with more or fewer components than shown here) disposed about the rotary or movable device.

FIGS. 2E-2I illustrate various examples of the impeller 506. The impeller 506 contains the driven magnet 538 which couples with the drive magnet 517 in the drive module 532. The driven or rotor magnet 538 can be pre-loaded and sealed into the impeller 506 so that the impeller 506 may be placed in the shroud 516 during assembly. As shown in FIG. 2I, the rotor or driven magnet 538 can be provided in a proximal portion of the impeller 506 and can be disposed about the impeller shaft 505. As explained herein, the drive magnet 517 can magnetically couple with the rotor magnet 538 to impart rotation to the rotor magnet 538 and, in turn, the impeller 506.

FIG. 3A is a schematic diagram of a power module 531A according to various embodiments. The power module 531A includes a plurality of components: a power lead 520A with an electrical connector 521A at a proximal portion, a set of contacts or pins 572A configured to fit into pre-existing sockets in the drive module 532, and an encasing member or second housing 529B′ which when fitted to and attached to the drive module 532 renders the pump 502 at least partially hermetically sealed and at least partially ionically sealed. FIG. 3A′ shows that in one variation, a power module 531A′ includes one or more sockets 569A configured to receive pins coupled with the motor housing 529A.

FIGS. 3B-3C illustrate another embodiment of a power module 531. In FIGS. 3B-3C, the power module can comprise a feedthrough body 570 having a disk 561 disposed in the housing 529B (also referred to herein as a lead contact housing). The disk 361 is relatively short, e.g., occupying only a fraction of the housing 529B. The disk 561 can occupy a portion of but less than half of the axial length of the housing 529B, less than a quarter of the axial length of the housing 529B, less than twenty percent of the axial length of the housing 529B. The disk 561 can be disposed distal to a volume 562 through which each of the conductors of the plurality of conductors 575 can be routed. As shown, an enclosure 563 can be disposed between the proximal end of the disk 361 and the distal end of the elongate body or lead 520. The enclosure 563 can be configured to at least partially surround the volume 562 through which each of the conductors 575 of the plurality of conductors 575 can be routed. In various embodiment, the enclosure 563 can comprise a polymeric body, such as a molding or potting compound.

As shown, the enclosure 563 can include a groove 564 configured to receive a seal member 566, such as an O-ring. The enclosure 563 can be disposed within the housing 529 as shown. In FIG. 3C, an adhesive 593 can be disposed between an outer surface of the enclosure 563 and an inner surface of the housing 529B. The adhesive 593 can be disposed within the enclosure 563 to control fluid ingress into the elongate body 520. For example, the volume 562 can be at least partially occupied by the adhesive 593 to control fluid ingress into the elongate body 520.

In FIGS. 3B-3C, a conductor passage assembly can include a support element (e.g., part of the disk 561 or a separate thin plate disposed on the proximal portion) and a plurality of tubular bodies 594. Each tubular body 594 can be configured to receive a distal portion of a conductor 575 of the plurality of conductors 575.

In various embodiments, the housing 529 can comprise a feature configured to be coupled with a motor housing enclosing a motor configured to be activated to produce rotational motion (e.g., to drive an impeller of a pump as explained herein). A connection portion can include a coupling surface and an electrical circuit (e.g., circuit board 568), with the electrical circuit being electrically connected to the plurality of pin receptacles (e.g., sockets 569), each pin receptacle or socket 569 can be configured to receive a pin of a plurality of pins 572 of the power lead to electrically connect the motor to the controller. In variations, the housing 519 can have sockets to mate with pins located on a drive module. The coupling surface can be configured to facilitate a sealing connection to the power lead, e.g., by way of a circumferential weld. In some embodiments, the pin receptable or socket 569 can be configured to electrically connect to a pin 572 of the power lead via a press-fit connection.

As explained herein, the pump 502 can beneficially be assembled modularly. For example, as explained herein, the power delivery module 531 can comprise an elongate lead body 530 having conductors 575 configured to convey power therethrough from a first end configured to be electrically connected to a controller to a second end. The second end of the power delivery module 531 can have a lead contact housing (e.g., a second housing 529B) supporting a first plurality of contacts, such as connector pins 572. The first plurality of contacts (e.g., pins 572 or sockets in variations) can be connected with a second plurality of contacts (e.g., sockets 569 or pins in variations) disposed on a first end portion 529A-1 of the motor housing (e.g., housing 529A) of the drive module 532. A free end 529B-1 of the lead contact housing 529B can be positioned adjacent to a first surface of the first motor housing 529A. A free end 516A of the shroud 516 of the hydraulic module 533 can be positioned adjacent to a second surface of the motor housing 529A. The lead contact housing 529B can be circumferentially joined to the motor housing 529A while maintaining a position and/or orientation of the lead contact housing 529B relative to the motor housing 529A. The free end 516A of the shroud 516 can be circumferentially joined to the motor housing 529A while maintaining a position and/or orientation of the shroud 516 relative to the motor housing 529A.

In various embodiments, the first plurality of contacts can comprise pins 572 and the second plurality of contacts can comprise sockets 569 (e.g., coupled with a circuit board). The pins 572 of the power delivery module 531 can be inserted into the sockets 569 of the drive module 532. Upon positioning the free end 529B-1 of the lead contact housing 529B against the first surface of the motor housing 529A, the pins 572 of the power module 531 can be in electrical contact with the sockets 569 of the drive module 532. In various embodiments, a longitudinal axis of the lead contact housing 529B can be aligned with a longitudinal axis of the motor housing 529A, e.g., using one or more jigs 537 as discussed above.

In various embodiments, a hermetic seal is provided for the drive module 532 by circumferentially joining the motor housing 529A to the lead contact housing 529B. An ionic seal can be provided for the lead contact housing 529B by circumferentially joining the motor housing 529A to the lead contact housing 529B. For example, in some embodiments, the lead contact housing 529B can be circumferentially joined to the motor housing 529A by providing relative motion between a welder and the lead contact housing 529B and the motor housing 529A while the position and/or orientation of the lead contact housing 529B and the motor housing 529A are maintained, such that the housings 529A, 529B are welded together circumferentially. In some embodiments, relative rotational motion between the welder and the lead contact housing 529B and the motor housing 529A is provided while the position and/or orientation of the lead contact housing 529B and the motor housing 529A are maintained. In some embodiments, circumferentially joining the motor housing 529B to the shroud 516 comprises providing relative rotational motion between a welder and the shroud 516 and the motor housing 529A while the position and/or orientation of the shroud 516 and the motor housing 529A are maintained, e.g., by the use of a jig 537. The jig 537 may apply a compressive force to maintain good contact between the free end 529B-1 of the lead contact housing 529B against a surface of the motor housing 529A opposite to the drive unit cover 511.

As explained herein, the blood pump 502 can have a width suitable for placement in an aorta of a patient. The elongate motor housing 529A and the lead contact housing 529B can have a diameter configured to permit substantial blood flow between an outer surfaces thereof and an aorta when deployed. For example, the elongate motor housing can comprise a diameter less than 2 cm.

The hydraulic module 533 can comprise an impeller 506 magnetically rotatable by operation of a motor 530 disposed in the drive module 532. The impeller 506 of the hydraulic module 533 can be positioned within the shroud 516. Positioning the free end of the shroud 516 adjacent to the second surfaced of the motor housing 529A can cause the rotor magnet 538 in the impeller 506 to be magnetically coupled with the drive magnet 517 in the drive module 532. A cover 511 of the motor housing 529A can also include a fixed projection that can serve as a bearing (similar to drive bearing 18 described herein). The impeller 506 can be disposed over the fixed projection or bearing when positioning the free end of the shroud 516 adjacent to the second surface of the motor housing 529A. In some embodiments, the cover 511 comprises a continuous surface without any openings for an output shaft of the motor 530. The output shaft 531 of the motor 530 can be coupled with the drive magnet 517 at a free end thereof. The drive magnet 517 can be separated from an inside surface of a cover of the drive module.

As noted above, the distal radial bearing 515 can be supported in the hydraulic module 533. To further assemble the hydraulic module 533, the impeller 506 can be positioned in the shroud 516. A distal portion of the impeller shaft 505 of the impeller 506 can slide into the distal radial bearing 515, e.g., into an inner sleeve 515A thereof when the inner sleeve 515A is provided, prior to positioning the free end of the shroud 516 of the hydraulic module 533 adjacent to the second surface of the motor housing 529A. In one method, a distal portion of the impeller shaft of the impeller 506 is inserted into the inner sleeve 515A. The shaft can rotate in the inner sleeve 515A forming a distal radial bearing for the impeller 506. The inner sleeve 515A can be supported on the peripheral outer sleeve of the distal radial bearing by a connecting structure 515B extending to the outer sleeve as shown in FIG. 2D′. Positioning the distal portion of the impeller shaft can be achieved by providing motion along the direction of the arrow Al in FIG. 2, as discussed above.

As explained herein, the hydraulic module 533 can include the strut module 580. The strut module 580 can be joined to the shroud 516 by forming at least one weld 592 between a distal portion of the shroud 516 and a sleeve 593 coupled with the strut module 580. A radial projection 584 of the sleeve 593 can engage with a recess 585 formed in a distal surface of the proximal ring 583 of the strut module 580 to couple the sleeve 593 with the strut module 580. The proximal ring 583 can include a continuous unperforated surface extending entirely around the proximal ring 583. The sleeve 593 can be disposed within the inner surface of the proximal ring 583 when coupled to the strut module 580 and extending to a position within the distal portion of the shroud 516.

A printed circuit board 568 can be positioned in a passage of the motor housing 529B, electrically connecting the second plurality of contacts 569 with the printed circuit board 568 (e.g., sockets 569 with the circuit board 568). The position of the second contacts 572 can be fixed adjacent to the first surface of the motor housing 529A. The second plurality of contacts can comprise press-fit receptacles or sockets 569 configured to be deflected by engagement thereof with the first plurality of contacts (e.g., the pins 572).

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1” includes “1.” Phrases preceded by a term such as “substantially,” “generally,” and the like include the recited phrase and should be interpreted based on the circumstances (e.g., as much as reasonably possible under the circumstances). For example, “substantially spherical” includes “spherical.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Although certain embodiments and examples have been described herein, it should be emphasized that many variations and modifications may be made to the humeral head assembly shown and described in the present disclosure, the elements of which are to be understood as being differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, it will be understood by those skilled in the art that the scope of the inventions extends beyond the specifically disclosed embodiments to any and all embodiments having equivalent elements, modifications, omissions, combinations or sub-combinations of the specific features and aspects of the embodiments (e.g., of aspects across various embodiments), adaptations and/or alterations, and uses of the inventions as would be appreciated by those in the art based on the present disclosure. The limitations in the claims are to be interpreted fairly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. A method for assembling a blood pump, comprising:
providing a power delivery module comprising an elongate lead body having conductors configured to convey power therethrough from a first end configured to be electrically connected to a controller to a second end, the second end having a lead contact housing supporting a first plurality of contacts;
coupling the first plurality of contacts with a second plurality of contacts disposed on a first end portion of a motor housing of a drive module;
positioning a free end of the lead contact housing adjacent to a first surface of the motor housing;
positioning a free end of a shroud of a hydraulic module adjacent to a second surface of the motor housing;
circumferentially joining the lead contact housing to the motor housing while maintaining a position and/or orientation of the lead contact housing relative to the motor housing; and
circumferentially joining the free end of the shroud to the motor housing while maintaining a position and/or orientation of the shroud relative to the motor housing.

2. The method of claim 1, wherein the first plurality of contacts comprises pins and the second plurality of contacts comprises sockets and wherein coupling the first plurality of contacts with the second plurality of contacts comprises inserting the pins of the power delivery module into the sockets of the drive module.

3. The method of claim 2, wherein maintaining the position and/or orientation of the lead contact housing relative to the motor housing comprises aligning a longitudinal axis of the lead contact housing with a longitudinal axis of the motor housing.

4. The method of claim 1, wherein circumferentially joining the lead contact housing to the motor housing comprises providing relative motion between a welder and the lead contact housing and the motor housing while the position and/or orientation of the lead contact housing and the motor housing are maintained.

5. The method of claim 1, wherein circumferentially joining the motor housing to the shroud comprises providing relative rotational motion between a welder and the shroud and the motor housing while the position and/or orientation of the shroud and the motor housing are maintained.

6. The method of claim 1, wherein the method provides a blood pump assembly having a width suitable for placement in an aorta of a patient.

7. The method of claim 1, wherein the hydraulic module comprises an impeller magnetically rotatable by operation of a motor disposed in the drive module.

8. The method of claim 7, further comprising positioning the impeller of the hydraulic module within the shroud and wherein positioning the free end of the shroud of the hydraulic module adjacent to the second surfaced of the motor housing causes a magnet in the impeller to be magnetically coupled with a magnet in the drive module.

9. A method for assembling a blood pump, comprising:
coupling a first plurality of contacts of lead contact housing of a lead assembly with a second plurality of disposed on a first end of a motor housing of a drive unit;
positioning a free end of the lead contact housing adjacent to a first surface of a motor housing; and
circumferentially joining the lead contact housing to the motor housing while maintaining a position and/or orientation of the lead contact housing relative to the motor housing.

10. The method of claim 9, wherein circumferentially joining comprises forming a laser weld about the periphery of the motor housing and the lead contact housing.

11. The method of claim 9, wherein a hermetic seal is provided for the motor housing by circumferentially joining the motor housing to the lead contact housing.

12. The method of claim 9, wherein an ionic seal is provided for the lead contact housing by circumferentially joining the motor housing to the lead contact housing.

13. The method of claim 9, further comprising:
positioning a free end of a shroud disposed around an impeller adjacent to a second surface of the motor housing; and
joining the free end of the shroud to the motor housing while maintaining a position and/or orientation of the shroud relative to the motor housing.

14. The method of claim 13, wherein joining comprises circumferential joining.

15. A method for assembling a medical device, comprising:
providing a power delivery module configured to delivery electrical power during operation of the medical device;
providing a drive module configured to convert the electrical power from the power delivery module to motion of a drive device during operation of the medical device;
providing a hydraulic module comprising a treatment device, the motion of the drive device imparting motion to the treatment device during operation of the medical device; and
connecting the drive module to the power delivery module and connecting the hydraulic module to the drive module.

16. The method of claim 15, further comprising connecting the drive module to the power delivery module before connecting the hydraulic module to the drive module.

17. The method of claim 15, wherein the drive device comprises a drive magnet.

18. The method of claim 15, wherein the treatment device comprises a pump.

19. The method of claim 15, wherein the motion comprises rotational motion.

20. The method of claim 15, wherein the motion comprises translational motion.
